(19) United States
(12) Patent Application Publication
(10) Pub. No.: US 2026/0294573 A1
(43) Pub. Date: Oct. 1, 2026

(54) DISSIMILAR POLYMER FILM JOINT WITH ENHANCED SEAL STABILITY FOR MEDICAL DRAPE

(71) Applicant: Medline Industries, LP, Northfield, IL (US)

(72) Inventor: Joshua D. Gunn, Marietta, GA (US)

(21) Appl. No.: 19/095,508

(22) Filed: Mar. 31, 2025

(51) Int. Cl. A61B 46/00; A61B 46/10

(52) U.S. Cl. CPC A61B 46/40; A61B 46/10

(57) ABSTRACT

A medical drape can be used during surgical procedures to help protect patients and equipment from contamination. The drape may comprise a first portion comprising a first polymer film material and a second portion comprising a second polymer film material. The two portions of film material may be connected at a joint. The joint may comprise an adhesive tape having an adhesive material on a first side to adhere the tape to the first film. A second side of the tape may be bonded or fused to the second film. The second side of the tape may be bonded or fused to the second film after the tape has been adhered to the first film.

### FIELD OF THE INVENTION

The present invention relates generally to surgical drapes for use, for example, in surgical theaters, and particularly to such drapes requiring joinder of different drape zones having different material properties.

### BACKGROUND

Medical drapes, also referred to as surgical drapes, can be used during surgical procedures to help protect patients and equipment from contamination. For example, a patient drape may be used to cover portions of a patient's body not involved in a surgical procedure, providing a window or segregated space for accessing a region of the patient's body on which the surgical procedure is being performed. As another example, an equipment drape can be used to cover portions of a piece of medical equipment used during a procedure. Example types of equipment that can be draped include imaging systems (e.g., C-arm imaging machines) and surgical equipment (e.g., robotic surgical systems). In either case, a drape can provide a physical barrier helping to protect the surgical field from contamination, protecting the patient, clinicians, and/or equipment. The appropriate use of medical drapes can help minimize the possibility of cross-contamination and hospital acquired infections between procedures and patients.

Surgical drapes can be made of a variety of different materials, including plastic, cloth, and paper. Surgical drapes can be designed for cleaning and reuse although more commonly are provided as a single-use disposable item. Each surgical drape used in a surgical environment may be designed for a specific purpose (e.g., covering certain patient anatomy, covering a specific piece of equipment). Accordingly, a surgical drape may be fabricated from a variety of different materials and/or have one or more accessory features connected thereto to configure the drape for its intended purpose. Individual drape components may typically be joined together using adhesive, heat welding, or the like. When working with drape components that have incompatibilities with each other (e.g., chemical incompatibility challenging adhesive bonding, thermal incompatibility challenging heat welding), there is an increased risk of subsequent joint failure between the components. If a surgical drape exhibits a joint failure during a medical procedure, there is risk of patient contamination from the environment, environmental/equipment contamination from the patient, and potential biohazard remediation requirements.

Various scenarios of surgical sterile draping applications require different material properties, guiding material selection for each drape. Many applications have different requirements over different areas or zones of the same drape. This may result, in particular, with dissimilar polymer films being required in different zones of the same drape which need to be sealed together in a way that maintains a sterile barrier over the entire shelf life of the product, which can extend for multiple years. Known methods of using adhesives to join dissimilar films often results in a joint that will separate, potentially creating channels through the sealed joint over time due to the differences in base materials. These channels constitute joint failure as it can no longer maintain sterility since liquid and particles can pass through.

Therefore, there is a need for improved joints between dissimilar materials in medical drapes that will produce a long-term stable sealed joint and for improved methods of creating such joints.

### SUMMARY OF ILLUSTRATIVE EMBODIENT

Embodiments of a method for forming a joint in a medical drape may be applied to a drape that comprises a first portion having a first edge section, where the first portion comprises a first polymer film. The first polymer film may comprise a first polymeric component and have a first melting temperature. The drape may further comprise a second portion having a second edge section, where the second portion comprises a second polymer film. The second polymer film may comprise a second polymeric component and have a second melting temperature that is lower than the first melting temperature. The drape may also comprise an adhesive tape. The adhesive tape may comprise a carrier strip that comprises the same second polymeric component as the drape second portion. The carrier strip may have a first side on which an adhesive layer is distributed and a second side that is free from adhesive.

The method of forming a joint between the two drape portions may comprise a first step of applying the tape to a surface of the first drape edge section such that the carrier strip first side is adhered to the first polymer film by the adhesive layer. After applying the tape to the first portion, a second step may comprise positioning the second drape portion such that the second edge section overlaps with the first edge section to form an overlap area with the adhesive tape positioned at least partially within the overlap area between the first edge section and the second edge section. Positioned in this manner, the carrier strip second side is adjacent, but not adhered, to the second polymer film. A third step may comprise applying heat to the overlap area such that the carrier strip second side is bonded to the adjacent second polymer film second edge section.

In further embodiments, the second polymeric film and the carrier strip materials may be compatible with heat bonding to each other. Conversely, the first polymeric strip and the carrier strip materials may be incompatible with heat bonding to each other.

In further embodiments, the second polymeric film and the carrier strip materials may be compatible with heat bonding to each other. Conversely, the first polymeric strip and the carrier strip materials may be incompatible with heat bonding to each other. The carrier strip may also have a third melting temperature that is lower than the first melting temperature. The first polymeric component may comprise a TPU material, and the second polymeric component may comprise an LDPE material.

In further embodiments of the method, at least a portion of the overlap area is heated to a temperature that is greater than the second melting temperature, while the temperature may be less than the first melting temperature. By embodiments of this method, the carrier strip second side is bonded to the adjacent second polymer film second edge section by fusing the second polymer film of the second drape portion with the second polymer film of the tape carrier strip.

In further embodiments of the method, the adhesive layer comprises a heat activated adhesive and applying heat may activate the adhesive. In addition, applying heat may enhance flow of the adhesive layer onto a surface of the first polymer film and a surface of the carrier strip first side. The step of applying heat may also comprise applying pressure to at least a portion of the overlap area. Applying pressure in addition to heat may flatten the overlap area and may make the joint more consistent and durable.

Further embodiments of a method for forming a joint in a medical drape may be applied to a drape that comprises a first portion having a first edge section, where the first portion comprises a first polymer film. The first polymer film may have a first melting temperature. The drape may further comprise a second portion having a second edge section, where the second portion comprises a second polymer film. The second polymer film may have a second melting temperature that is lower than the first melting temperature. The drape joint may also comprise an adhesive tape. The adhesive tape may comprise a carrier strip, which may comprise a third polymer film. The carrier strip may have a third melting temperature that is lower than the first melting temperature. In addition, the carrier strip may comprise a first side on which an adhesive layer is distributed and a second side that is free from adhesive.

The method of forming a joint between the two drape portions may comprise a first step of applying the tape to a surface of the first drape edge section such that the carrier strip first side is adhered to the first polymer film by the adhesive layer. After applying the tape to the first portion, a second step may comprise positioning the second drape portion such that the second edge section overlaps with the first edge section to form an overlap area with the adhesive tape positioned at least partially within the overlap area between the first edge section and the second edge section. Positioned in this manner, the carrier strip second side is adjacent, but not adhered, to the second polymer film. A third step may comprise applying heat to the overlap to raise the temperature of at least a portion of the overlap area to a temperature that is lower than the first melting temperature and higher than the second and third melting temperatures.

In further embodiments, heating the overlap area may cause the carrier strip second side to bond to the adjacent second polymer film second edge section. This bonding may be by fusing the second polymer film of the second drape portion with the carrier strip. In some embodiments, the second polymer film may comprise a polymeric component, and the carrier strip may comprise the same polymeric component.

In additional embodiments, the melting temperature of the first portion film is higher than 135º C, and the melting temperature of the second portion and carrier strip films are lower than 130º C. Alternatively, the melting temperature of the first portion film is higher than 150º C, and the melting temperature of the second portion and carrier strip films are lower than 150º C. In further alternatives, the melting temperature of the first portion film is higher than 150º C, and the melting temperature of the second portion and carrier strip films are lower than 130º C.

Embodiments of a medical drape manufactured in a manner that is consistent with the present disclosure may comprise a first portion comprising a first polymer film with a first melting temperature and a second portion comprising a second polymer film with a second melting temperature that is lower than the first melting temperature. An adhesive tape comprising a carrier strip may form a joint between the two films. The carrier strip may comprise a third polymer film, with a third melting temperature that is lower than the first melting temperature. The carrier strip may also have a first side on which an adhesive layer is distributed and a second side that is free from adhesive.

The tape may be applied to a surface of the first drape portion such that the carrier strip first side is adhered to the first polymer film by the adhesive layer. The second drape portion may then be positioned such that it overlaps with the first drape portion in an overlap area with the adhesive tape positioned in the overlap area such that the tape carrier strip second side is adjacent to the second polymer film. The second polymer film of the second drape portion may then be fused with the third polymer film of the carrier strip to join the two drape portions together. The first polymer film may comprise a TPU material, while the second and third polymer films may comprise LDPE materials.

### BRIEF DESCRIPTION OF THE DRAWINGS

The foregoing and other advantages of the invention will become apparent upon reading the following detailed description and upon reference to the drawings.

FIG. 1 shows an embodiment of a medical drape.

FIG. 2 shows a schematic view of a first embodiment of a medical drape.

FIG. 3 shows a schematic view of a further embodiment of a medical drape.

FIG. 4 shows a schematic view of a process for manufacturing embodiments of the medical drape of FIG. 3.

FIG. 5 shows a diagram illustrating steps in a process for manufacturing embodiments of the medical drape of FIG. 3.

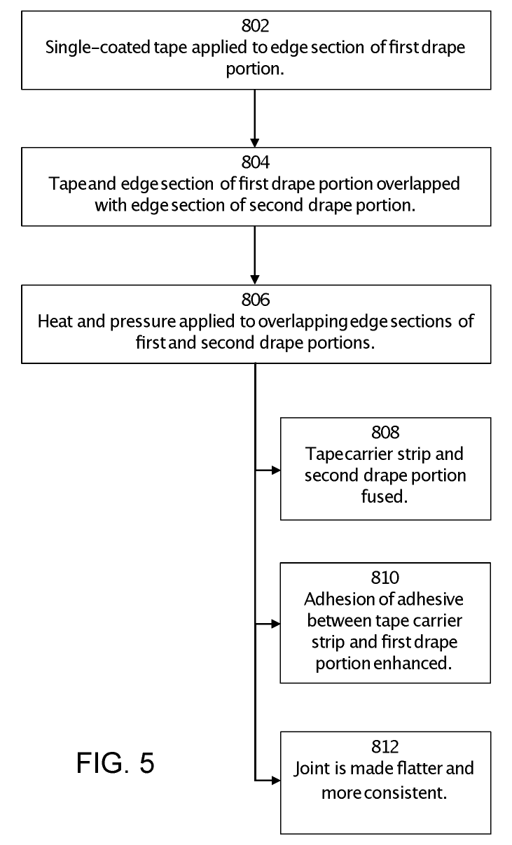

The drawings listed above are intended to convey to one of ordinary skill in the art the present invention and its embodiments. In some drawings certain elements have not been shown for clarity. While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. The following description of various embodiments, as represented in the figures, is not intended to limit the scope of the present disclosure but is merely representative of various embodiments. Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Referring to the drawings, like numbers generally indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, proximal and distal, posterior and anterior, inner and outer, and the like may be used solely to distinguish one entity or action from another entity or action as illustrated in the figures or otherwise without necessarily requiring or implying any actual such relationship or order between such entities or actions.

Additionally, instances in this specification where one element is “coupled to another element” can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Coupling, and securing may comprise various mechanism, including, but not limited to, adhesive, tape, bonding, or integral formation. Additionally, as used herein, “adjacent” does not necessarily denote contact. For example, one element can be adjacent to another element without being in contact with that element. In addition, the term “clinician” is used herein to indicate the medical practitioner or any other person that may utilize equipment or conduct procedures as described herein. The term includes doctors, nurses, attendants, or other medical personnel and is not intended to limit the scope of the described or claimed embodiments.

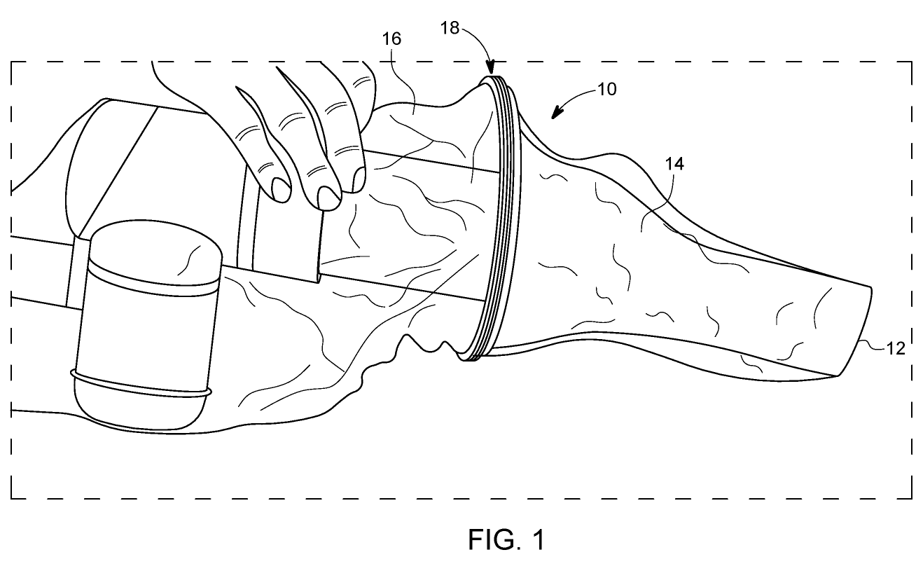

FIG. 1 is an illustration of an example medical drape 10 according to the disclosure. Drape 10 is illustrated as being implemented as a robotic surgical arm drape configured to be positioned over a surgical arm 12 of a robotic surgical system. Drape 10 can have a variety of different configurations and implementations. As shown in FIG. 1, drape 10 can be formed from at least two physically separate sections of drape material, which is illustrated as including a first section of drape material 14 and a second section of drape material 16, that are joined together to form a combined structure (e.g., unity structure, integral structure). First section of drape material 14 and second section of drape material 16 are joined together by a joint 18. Joint 18 can connect portions of first section of drape material 14 and second section of drape material 16 together, forming a closed (e.g., sealed) joint line between the two sections of material.

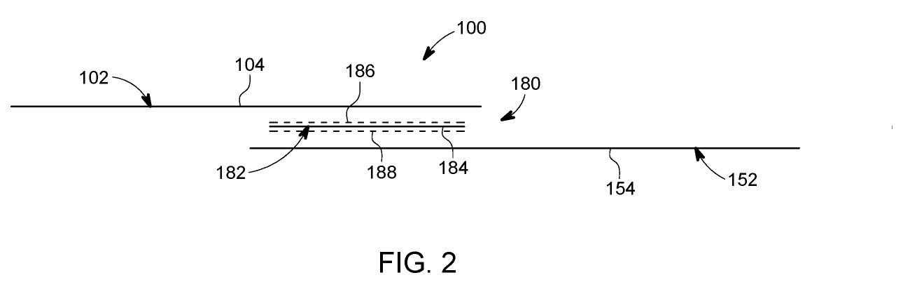

FIG. 2 illustrates a joint 180 between portions of a medical drape 100. A first portion 102 may comprise a first polymer film 104. The first polymer film may comprise a first polymeric component. The first polymeric component may be characterized by its physical or chemical properties. The first polymeric component may comprise the primary polymeric component of the first film, which may also comprise one or more secondary polymeric or other components. For example, the first polymer film may comprise a film formed with thermoplastic polyurethane (“TPU”) as the primary polymeric component. The first film may have a thickness between one mil and five mils with the thickness preferably being approximately two mils.

A second portion 152 may comprise a second polymer film 154. The second polymer film may comprise a second polymeric component. The second polymeric component may be characterized by its physical or chemical properties, and these physical or chemical properties may differ from those of the first polymeric component. In particular, the second polymeric component may have a melting temperature that differs from the melting temperature of the first polymeric component. The second polymeric component may comprise the primary polymeric component of the second film, which may also comprise one or more secondary polymeric or other components. For example, the second polymer film may comprise a film formed with low-density polyethylene (“LDPE”) as the primary polymeric component. The second film may have a thickness between one mil and five mils with the thickness preferably being approximately two mils.

The joint 180 may comprise a lap joint that is secured by a double-coated adhesive tape 182. The tape 182 may comprise a carrier strip 184. The carrier strip may comprise a material that is different from the first film 104 and the second film 154. The carrier strip 184 may comprise a polyethylene terephthalate (“PET”). Alternatively, the carrier strip 184 may comprise a material other than PET but that is compatible with an adhesive that will also adhere to the material of the first 104 and second 154 drape films.

The adhesive tape 182 may further comprise a first layer of adhesive 186 that is positioned between the carrier strip 184 and the first drape film 104. The first adhesive 186 may comprise a first adhesive that is capable of bonding the carrier strip 184 with the first drape film 104. In embodiments of the joint 180 illustrated in FIG. 2, the first adhesive may be an adhesive that is capable of bonding TPU of the first drape portion 104 with PET of the carrier strip 184. The adhesive tape 182 may further comprise a second layer of adhesive 188 that is positioned on an opposite side of the carrier strip 184 between the carrier strip and the second drape film 154. The second adhesive 188 may comprise a second adhesive that is capable of bonding the carrier strip 184 with the second drape film 154. In embodiments of the joint 180 illustrated in FIG. 2, the second adhesive may be an adhesive that is capable of bonding LDPE of the second drape portion 154 with PET of the carrier strip 184. The first 186 and second 188 adhesives may comprise the same adhesive or may comprise different adhesives.

Accordingly, as illustrated in FIG. 2, the layers of illustrative embodiments of the joined drape portions are: 1) TPU first film 104; 2) first adhesive 186; 3) PET carrier strip 184; 4) second adhesive 188; and 5) LDPE second film 154. The dissimilar material properties of these elements, including properties such as thermal coefficient of expansion, surface energy, and others, may cause the joint layers to perform different on either side of the joint, and the difference in movement from the first TPU film 104 to the second LDPE film may be sufficient to cause separations in the adhesive bond. Accordingly, embodiments of the joint illustrated in FIG. 2 may exhibit reduced stability over time as the joint may fail to maintain a good seal over the expected usable life of the drape.

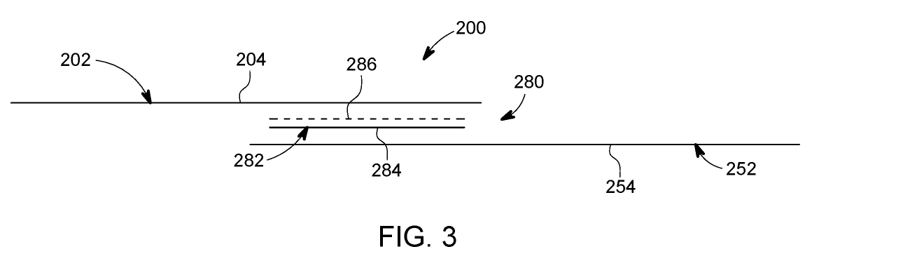

FIG. 3 illustrates embodiments of a joint 280 between portions of a medical drape 200. A first drape portion 202 may comprise a first polymer film 204. The first polymer film may comprise a film formed with thermoplastic polyurethane (“TPU”) as the primary polymeric component. The first film may have a thickness between one mil and twenty mils with the thickness preferably being approximately two mils. A second drape portion 252 may comprise a second polymer film 254. The second polymer film may comprise a film formed with low-density polyethylene (“LDPE”) as the primary polymeric component. The second film may have a thickness between one mil and twenty mils with the thickness preferably being approximately two mils.

The joint 280 may comprise a lap joint that is secured by a single-coated adhesive tape 282. The tape 282 may comprise a carrier strip 284. The carrier strip may comprise a material that is different from the first film 204. However, the carrier strip 284 may comprise a material that is the same as or similar to the second film 254. For example, the carrier strip 284 may comprise an LDPE material. Alternatively, the carrier strip 284 may comprise a material that is distinct from the second drape film 254 material but that may be thermally or ultrasonically bonded with the second drape film material. For example, the carrier strip 284 may comprise a material other than LPDE but that that may be bonded with LPDE under heat and pressure conditions, and more preferably heat and pressure conditions that are insufficient to melt or bond with TPU material such as the TPU material of the first drape portion 202. One non-limiting example of a suitable tape is Medical Tape 1525L Single Sided Transparent Polyethylene Medical Tape (3M).

As discussed above, the joint may be used to join thermoplastic materials having significantly different melting temperatures such as a TPU material and an LPDE material. Alternatively, there may be a need to join thermoplastic materials such as an LPDE material with materials that do not readily melt, and as such are not amenable to thermal, ultrasonic, or other bonding requiring melting. Accordingly, embodiments of the joint 280 contemplate joining a first portion 202 that comprises a first film 204 comprising a thermoset or other material that does not readily melt. For example, the first film 204 may comprise a thermoset material such as dip-molded polyisoprene. Such a material may not amenable to heat bonding, and known adhesive methods of bonding may be susceptible to channeling due to the elastomeric nature of the polyisoprene material.

Returning to FIG. 3, the adhesive tape 282 may further comprise a layer of adhesive 286 that is positioned between the carrier strip 284 and the first drape film 204. The adhesive 286 may comprise a first adhesive that is capable of bonding the carrier strip 284 with the first drape film 204. In embodiments of the joint 280 illustrated in FIG. 3, the first adhesive may be an adhesive that is capable of bonding TPU of the first drape portion 204 with LDPE of the carrier strip 284. Is discussed above, the adhesive tape 282 may be thermally bonded to the second drape film 254 on an opposite side of the carrier strip 284 from the adhesive 286 and first drape film 204. The carrier strip 284 and second film 254 may be bonded by the application of heat and pressure to an interface between the carrier strip and the second film such as provided, for example, by an impulse sealing machine or similar method or device. Alternatively, the carrier strip and second film may be bonded by ultrasonic bonding or other bonding methods without the use of adhesive as would be known by one of ordinary skill in the art.

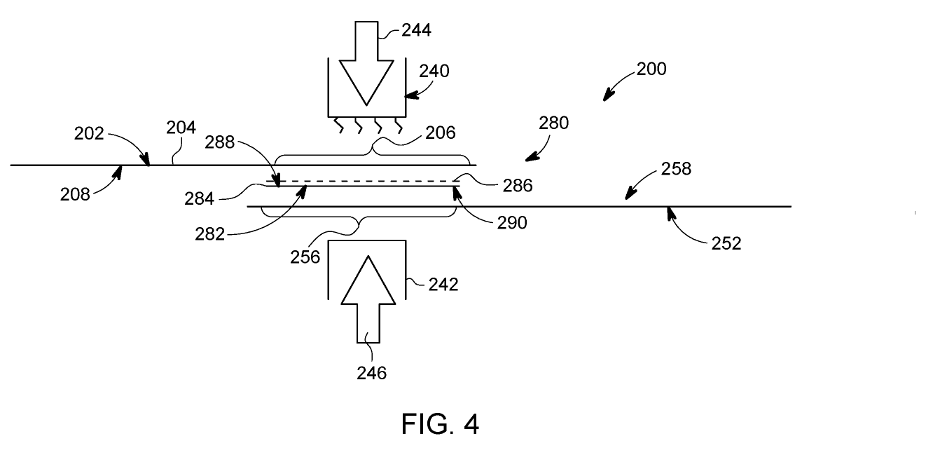

FIGS. 4 & 5 illustrate a method of manufacturing a medical drape comprising two or more sections of disparate film materials joined by a joint. The drape 200 may comprise a first drape section 202 comprising a first film material 204. The first drape section 202 is joined to a second drape section 252 comprising a second film material 254. The second film material has a different composition from the first film material.

Embodiments of a joining process 800 may comprise a number of steps. In the embodiments illustrated of the drape and joining method illustrated in FIGS. 4 & 5, a first film 204 comprising a TPU material is joined to a second film 254 comprising a LPDE material by the exemplary method. However, it will be understood by one of ordinary skill in the art that the structure and steps described with regard to these materials may also apply to other materials. Additional or alternative steps may also be used to the extent that such steps do not directly interfere with embodiments of the general method as described herein. In addition, when discussing materials that are the “same” or “different” it will be understood that the materials need not be chemically identical but that same or different refer to characteristics that are relevant to the properties or behavior being specifically discussed with regard to embodiments of a medical drape.

In a first step 802, a single-coated adhesive tape 282 is overlapped with and applied to an edge section 206 of a first film 204 of a first drape portion 202. The first film may comprise a TPU material, and the adhesive tape may comprise carrier strip 284 comprising a LDPE material. The LDPE material of the carrier strip may have a melting or tackifying temperature that is different from the TPU material of the first film. Preferably, the melting temperature of the LDPE material is lower than the melting temperature of the TPU material.

The first film may have a melting temperature between 135º C and 220º C, preferably between 150º C and 200º C, and more preferably between 160º C and 185º C. The second film and carrier strip may both have melting temperatures between 100º C and 150º C, preferably between 105º C and 130º C, and more preferably between 105º C and 115º C. The melting temperatures of the two may be the same or different but within the ranges specified.

The tape may also comprise an adhesive layer 286 positioned on a topside 288 of the carrier strip. In the first step 802, the adhesive layer 286 is brought into contact with a lower surface 208 of the first film 204. Following this first step, the single-coated tape 282 and its LDPE carrier strip 284 is adhered to the TPU film 204 of the first drape portion 202.

In a second step 804, the edge section 206 of the first film, including the attached tape 282, is overlapped with an edge section 256 of a second film 254 of a second drape portion 252. The second film may comprise an LDPE material that is the same as or compatible with the LDPE material of the tape carrier strip. The second film LDPE material may have a melting temperature that is similar to or the same as the tape carrier LDPE material. Preferably, the melting temperature of the LDPE materials of both the tape carrier 284 and second film 254 are lower than the melting temperature of the TPU material of the first film 204. A bottom surface 290 of the tape carrier strip 284 is positioned adjacent to a top surface 258 of the second drape film 254. Following this second step, the tape 282 is adhered to the first drape portion 202 but not the second drape portion 252.

In a third step 806, heat and pressure are applied to the overlapping edge sections 206, 256 of the first and second films 204, 254, respectively, with the tape 282 sandwiched between the edge sections. The temperature that is applied is a predetermined temperature that is greater than a melting temperature of the LDPE materials of the tape carrier 284 and the second film 254 but less than a melting temperature of the TPU material of the first film 204. Accordingly, the predetermined temperature is sufficient to at least partially melt or tackify the LDPE materials but not the TPU material. Heat and pressure may be applied by one or more plattens 240, 242 of an impulse sealer. The pressure may be applied in opposing directions 244, 246 perpendicular to a plane of the drape. The heat and pressure may be applied in discrete sections or may be applied by an appropriate mechanism, such as a roller, during a continuous application during a manufacturing process in which the drape moves continuously through a sealing station of a manufacturing line.

In embodiments of the method, heat and pressure applied in the third step 806 may provide one or more effects or results 808, 810, 812 relative to the first TPU film 204, the second LDPE film 254, and the tape carrier 284. In a first result 808, the bottom surface 290 of the carrier strip 284 and the top surface 258 of the second film 254 may be melted and fused together in at least a portion of the edge section 256. In a second result 810, the heat and pressure application may enhance the adhesion provided by the adhesive 286 between the first TPU film 204 and the tape carrier 284. The heat and pressure may enhance flow of the adhesive onto the surfaces of the film 204 and tape carrier 284, including possible advantages of filling in all available spaces and maximizing surface contact. Finally, in a third result 812, the flatness and consistency of the joint 280 may be enhanced by an “ironing” effect of the application of the heat and pressure.

Consistent with embodiments of the drape and joining method described herein, when joining two films having different temperature characteristics, by using a single coated adhesive tape and matching heat process temperature to the lower melting temperature material, a heat-activated adhesive bond may be maximized to the higher melting temperature material, TPU for example. And the single coated tape may be directly bonded or welded to the lower melting temperature material, LDPE for example. Accordingly, embodiments of the joined drape and associated manufacturing method may create a higher strength, more consistent bond than achieved by known joining methods. In addition, the joint may be more capable of withstanding extended shelf life without channeling or other seal failures.

The written description set out above describes various features and aspects of various embodiments of drapes for use with equipment in a surgical theater or other situation requiring sterile covering. However, it will be understood that the accompanying figures may illustrate additional features, elements, or functionality that, while not explicitly described, would be understood by a person having ordinary skill in the art. The figures and any features, elements, or functionality that would be understood therefrom form part of the disclosure of this application. While the present invention has been described with reference to one or more particular embodiments, those skilled in the art will recognize that many changes may be made thereto without departing from the spirit and scope of the present invention. Furthermore, components from one embodiment can be used in other non-exclusive embodiments. Each of these embodiments and obvious variations thereof is contemplated as falling within the spirit and scope of the invention.

What is claimed is:

1. A method for forming a joint in a medical drape, the drape comprising
a first portion having a first edge section and comprising a first polymer film, the first polymer film comprising a first polymeric component and having a first melting temperature;
a second portion having a second edge section and comprising a second polymer film, the second polymer film comprising a second polymeric component and having a second melting temperature that is lower than the first melting temperature; and
an adhesive tape comprising a carrier strip, the carrier strip comprising a first side on which an adhesive layer is distributed and a second side that is free from adhesive, wherein the carrier strip comprises the second polymeric component;
the method comprising the steps of:
applying the tape to a surface of the first drape edge section such that the carrier strip first side is adhered to the first polymer film by the adhesive layer;
positioning the second drape portion such that the second edge section overlaps with the first edge section to form an overlap area and the adhesive tape is positioned at least partially within the overlap area between the first edge section and the second edge section with the carrier strip second side adjacent to the second polymer film; and
applying heat to the overlap area such that the carrier strip second side is bonded to the adjacent second polymer film second edge section.

2. The method of claim 1 wherein the second polymer film and the carrier strip are compatible with heat bonding to each other.

3. The method of claim 2 wherein the first polymer film and the carrier strip are incompatible with heat bonding to each other.

4. The method of claim 1 wherein the carrier strip has a third melting temperature that is lower than the first melting temperature.

5. The method of claim 4 wherein the step of applying heat heats at least a portion of the overlap area to a temperature that is greater than the second melting temperature and greater than the third melting temperature.

6. The method of claim 5 wherein the step of applying heat heats at least a portion of the overlap area to a temperature that is less than the first melting temperature.

7. The method of claim 1 wherein the carrier strip second side is bonded to the adjacent second polymer film second edge section by fusing the second polymer film of the second drape portion with the carrier strip.

8. The method of claim 1 wherein the first polymeric component comprises a TPU material.

9. The method of claim 1 wherein the second polymeric component comprises an LDPE material.

10. The method of claim 1 wherein the adhesive layer comprises a heat activated adhesive.

11. The method of claim 10 wherein the step of applying heat activates the adhesive.

12. The method of claim 1 wherein the step of applying heat enhances flow of the adhesive layer onto a surface of the first polymer film and a surface of the carrier strip first side.

13. The method of claim 1 wherein the step of applying heat also comprises applying pressure to at least a portion of the overlap area.

14. The method of claim 13 wherein the step of applying heat flattens the overlap area.

15. A method for forming a joint in a medical drape, the drape comprising
a first portion having a first edge section and comprising a first polymer film, the first polymer film having a first melting temperature;
a second portion having a second edge section and comprising a second polymer film, the second polymer film having a second melting temperature that is lower than the first melting temperature; and
an adhesive tape comprising a carrier strip having a third melting temperature that is lower than the first melting temperature, the carrier strip comprising a first side on which an adhesive layer is distributed and a second side that is free from adhesive;
the method comprising the steps of:
applying the tape to a surface of the first drape edge section such that the carrier strip first side is adhered to the first polymer film by the adhesive layer;
positioning the second drape portion such that the second edge section overlaps with the first edge section to form an overlap area and the adhesive tape is positioned at least partially within the overlap area between the first edge section and the second edge section with the carrier strip second side adjacent to the second polymer film; and
heating at least a portion of the overlap area to a temperature that is lower than the first melting temperature and higher than the second melting temperature and third melting temperature.

16. The method of claim 15 wherein heating the overlap area causes the carrier strip second side to bond to the adjacent second polymer film second edge section.

17. The method of claim 16 wherein the carrier strip second side is bonded to the adjacent second polymer film second edge section by fusing the second polymer film of the second drape portion with the carrier strip.

18. The method of claim 15 wherein second polymer film comprises a polymeric component and the carrier strip comprises the polymeric component.

19. . The method of claim 15 wherein the first temperature is higher than 135 ℃, the second temperature is lower than 130 ℃, and the third temperature is lower than 130 ℃.

20 . The method of claim 15 wherein the first temperature is higher than 150 ℃, the second temperature is lower than 150 ℃, and the third temperature is lower than 150 ℃.

21 . The method of claim 20 wherein the first temperature is higher than 150 ℃, the second temperature is lower than 130 ℃, and the third temperature is lower than 130 ℃.
